(19) United States
(12) Patent Application Publication
(10) Pub. No.: US 2024/0299272 A1
(43) Pub. Date: Sep. 12, 2024

(54) PERSONAL CARE COMPOSITION

(71) Applicant: The Procter & Gamble Company, Cincinnati, OH (US)

(72) Inventors: Debora W. Chang, Mason, OH (US); Brandon Scott Lane, Hamilton, OH (US); Maria Eugenia Gomez, Lebanon, OH (US); Joshua Mikell Williams, Cincinnati, OH (US)

(21) Appl. No.: 18/599,717

(22) Filed: Mar. 8, 2024

Related U.S. Application Data
(60) Provisional application No. 63/489,023, filed on Mar. 8, 2023.

(51) Int. Cl. A61K 8/46; A61K 8/44; A61K 8/49; A61Q 19/00

(52) U.S. Cl. CPC A61K 8/463; A61K 8/442; A61K 8/4926; A61Q 19/00; A61K 2800/10; A61K 2800/596

(57) ABSTRACT

A personal care composition that includes an acyl taurate surfactant and exhibits improved foaming and lathering in hard water. The surfactant system in the personal care composition also includes an N-alkyl acyl taurate surfactant and/or an amphoteric co-surfactant. The weight ratio of acyl taurate to N-alkyl acyl taurate and/or co-surfactant is tailored to provide the desired hard water lather benefit. The surfactant system also permits viscosity building with inorganic salt and demonstrates good lather and foaming when a cationic conditioning polymer is added.

### FIELD

The present disclosure generally relates to a personal care composition comprising an acyl taurate surfactant and an N-alkyl acyl taurate surfactant. More specifically, the present disclosure relates to personal care compositions comprising a combination of acyl taurate surfactant and N-alkyl acyl taurate surfactant and/or co-surfactant that provides improved lather in hard water.

### BACKGROUND

Human hair and skin become soiled due to contact with the surrounding environment and from sebum secreted by the skin or scalp. Soiled hair, in particular, can have an undesirable feel and/or appearance. Soiled hair and skin are typically cleaned with water and a surfactant-containing personal care composition such as a shampoo or body wash. When using the personal care composition, many consumers desire good lather. Indeed, the quantity and/or quality of lather is commonly associated with the performance of the personal care product. For example, a shampoo that provides lots of rich, creamy lather may be perceived as providing superior hair cleaning compared to a shampoo that provides less lather or less creamy lather. And it has been found that consumers have a more enjoyable cleaning experience when using products that generate more lather.

A factor that can impact the quantity and/or quality of lather produced by a personal care composition is water hardness. Hard water is characterized by a relatively high dissolved mineral content, especially calcium and magnesium. Water hardness may be based on the content of calcium carbonate. For example, more than 7.5 grains of calcium carbonate per gallon of water (gpg) is considered hard, whereas 0-3 gpg is considered soft. One grain is equal to 56.7 milligrams (0.002 ounces) of calcium carbonate. Water hardness may also be based on the level of total dissolved solids (TDS) in the water. For example, a TDS of over 120 ppm is typically considered hard, whereas a TDS of 60 or less is typically considered soft. Water with a TDS of 61-120 may be considered moderately hard, and exhibit characteristics of soft or hard water, depending on where it lies on the scale.

Using a person care composition such as a shampoo, conditioner or body wash in hard water tends to result in less lather production, as compared to soft water. It is generally understood that dissolved minerals in water, especially calcium, form metal ions in the water that can bind to the negatively charged portions of the surfactant in the personal care composition, which reduces the amount or quality (richness) of the lather. The metal ions can also reduce the ability of the surfactant to bind and remove dirt and oil, which negatively impacts product performance. Thus, cleansing compositions like shampoos, conditioners and body washes tend to work better (e.g., provide the best lather) in soft water.

In the U.S., it is estimated that 85% of people do not have soft water, and this figure may be higher worldwide. Thus, there is a need for cleansing compositions that provide suitable lather when used in hard water.

Most personal care compositions (e.g., shampoos and body washes) are surfactant-based compositions. That is, the composition uses one or more surfactants to provide a cleaning benefit (detersive surfactant), solubility benefit (co-surfactant), or other benefit (e.g., conditioning benefit). Sulfated surfactants are generally very good at removing oil and other contaminants from hair and skin, but they also have drawbacks. For example, sulfated surfactants are sometimes associated with poor quality hair feel, hair dryness and/or skin dryness after washing. This is commonly referred to as “harshness,” and harsh cleansing compositions can face poor consumer acceptance. Thus, there is a need to provide a milder surfactant system that provides good cleansing benefits as well as overcoming the foam and lather problems associated with hard water.

A variety of relatively mild surfactants are known for use in personal care compositions. For example, N-alkyl acyl taurates such as sodium methyl cocoyl taurate (SMCT) and sodium lauroyl taurate (SLT) are known, and generally exhibit suitable solubility in water (e.g., compared to sodium cocoyl isethionate), and do not hydrolyze at acidic pH. However, the alkyl methyl group bonded to the amide nitrogen in SMCT and SMLT can make it difficult to build viscosity in an aqueous personal care composition by the addition of an inorganic salt, which is typically how viscosity is built in a surfactant-based compositions. Without sufficient viscosity, it can be difficult to dispense and apply the composition in a controlled manner, and the composition may be perceived by a consumer as being low quality.

Another drawback of removing sulfated surfactants is the difficulty of adding a cationic conditioning polymer. Conditioning shampoos (i.e., shampoos that provide a cleansing and conditioning benefit to hair) typically include an anionic detersive surfactant and a cationic condition polymer. The cationic conditioning polymers form a coacervate with an anionic surfactant system during the intended use of the composition, which deposits on hair to provide the easier wet combing and detangling benefit desired by consumers. However, the formation of a coacervate in the product prior to use ties up a portion of the surfactant and decreases the amount of surfactant available to provide foaming and cleansing. Since, non-sulfated surfactants tend to be less effective than sulfated surfactants at foaming and cleansing, the addition of a cationic polymer can further tax those sulfate free surfactant systems and result in additional decreases in foaming and cleansing.

Accordingly, it would be desirable to provide a personal cleansing composition that provides suitable lather and cleaning performance in hard water. It would also be desirable to provide a personal cleansing composition that includes a milder surfactant with suitable solubility, stability, foaming and cleansing properties. It would further be desirable to provide a personal cleansing composition with a milder surfactant that allows for the thickening of the composition by the addition of an electrolyte such as an inorganic salt. It would still further be desirable to provide a personal care composition with a milder surfactant and a cationic polymer that exhibits good foaming and cleansing.

### SUMMARY

Disclosed herein is an aqueous personal care composition comprising a detersive surfactant comprising an acyl taurate surfactant or a combination of an acyl taurate and an N-alkyl acyl taurate surfactant, and an amphoteric co-surfactant, wherein a ratio of detersive surfactant to co-surfactant is about 0.5:1 to about 2:1.

### DETAILED DESCRIPTION

Recent trends indicate a desire by consumers to replace their sulfated cleansing compositions with milder, sulfate-free versions. However, conventional sulfate-free personal care compositions are commonly perceived as less effective due to, at least in part, to poor foaming and lathering properties compared to their sulfated counterparts. To make matters worse, using personal care compositions in hard water further inhibits foam and lather generation. Surprisingly, it has now been found that combining an acyl taurate surfactant and an N-alkyl acyl taurate surfactant and/or an amphoteric co-surfactant can provide desirable foaming and lather properties, even in hard water. It has further been found that these combinations of surfactants can be combined with a cationic polymer (e.g., cationic conditioning polymers) with little or no undesirable impact on foam properties.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

### Personal Care Composition

The sulfate-free personal care compositions herein include a detersive taurate surfactant for cleaning a target bodily surface such as hair and skin. In some instances, the personal care composition may include a co-surfactant, for example, to help solubilize the detersive surfactant or another ingredient in the composition. The taurate surfactant includes an acyl taurate surfactant and, optionally, an N-alkyl acyl taurate surfactant (e.g., N-methyl, N-ethyl, N-propyl or N-butyl acyl taurate surfactant). The personal care composition may also include a cationic polymer to aid in the appearance and/or feel of hair (i.e., a conditioning polymer).

It has surprisingly been found that when a suitable acyl taurate surfactant is combined with a suitable N-alkyl acyl taurate surfactant and/or amphoteric co-surfactant, improved lather is observed in hard water. This hard water lather benefit is also exhibited when a cationic conditioning polymer is added. In some instances, the combination of acyl taurate surfactant and N-alkyl acyl taurate surfactant can be tailored to provide a solubility and/or stability benefit.

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, conditioning benefit to hair in combination with a detersive taurate surfactant.

Liquid personal care compositions herein, such as shampoos, conditioners, and body washes may have a viscosity of 2,000 mPa-s to 20,000 mPa-s (e.g., 2,500-15,000 mPa-s, 3,000-10,000 mPa-s or 3,500-9,000 mPa-s) according to the Rheology method described in more detail below. It is believed that viscosities in this range are generally preferred by users of liquid personal care compositions.

In some aspects, the compositions herein may contain an inorganic salt thickener such as sodium chloride, potassium chloride, sodium sulfate, ammonium chloride, sodium bromide, combinations of these and the like. In some aspects, the inorganic salt may be present at 0-2%, (e.g., 0.05-1% or 0.1-0.5%). In some sulfate-free cleansing compositions, inorganic salt can introduce instability to the composition by aiding in the formation of a coacervate between anionic surfactants and cationic polymers that may be present. This is typically perceived as composition instability. The coacervate typically has a gel-like consistency, which can precipitate when the composition is unstable, and it can impact the rheological and performance properties of the composition as well as the consumer-perceived quality of the personal care product. Thus, it can be important to specifically tailor the amount of inorganic salt in the composition formulation. Too little salt may result in a composition with insufficient viscosity, and too much salt may result in an unstable product.

Of course, it is to be appreciated that when the cleansing composition is used as intended, it will form a coacervate upon dilution to provide the desired cleaning and/or conditioning benefit.

### Taurate Surfactant

The sulfate-free surfactant system of the personal care compositions herein includes an acyl taurate surfactant and, optionally, an N-alkyl acyl taurate surfactant to provide foaming and cleaning properties to the personal care composition. Such surfactants are generally referred to as detersive surfactants. Detersive surfactants facilitate cleaning due to their amphiphilic nature, which allows the surfactants to break up, and form micelles around, oil and other contaminants on the hair. The detersive surfactant also produces lather, which helps lift “entrapped” contaminants off the hair or skin to be rinsed off more easily with water.

The composition may include 1-20% of taurate surfactant (e.g., 2-15%, 3-12%, or 4-10%). The taurate surfactant may include a weight ratio of acyl taurate to N-alkyl acyl taurate of 1:4 to 50:1 (e.g., 1:4, 1:3, 1:2, 1:1, 2:1, 3:1, 4:1 5:1, 6:1, 7:1, 8:1, 9:1, 10:1, 11:1, 12:1, 13:1, 15:1 20:1, 25:1, 30:1, 35:1, 40:1 or 45:1). It can be important not to include too much N-alkyl acyl taurate, as it can interfere with the ability to build viscosity in the composition, for example, using inorganic salt such as sodium chloride and/or potassium chloride. Compositions with insufficient viscosity (i.e., “runny”) may be viewed as poor quality by a consumer.

Acyl taurate surfactants that may be suitable for use herein are generally described by Formula I illustrated below.

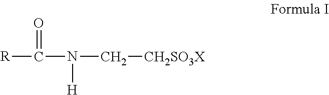

Where: R is an alkyl group with 5 to 23 carbon atoms (7-21, 7-17, 7-15, 7-13, 11-17, 11-15, 11-13 or even 11 carbon atoms) and X is a suitable counterion (e.g., sodium, potassium, magnesium, ammonium or triethanolamine).

Some nonlimiting examples of acyl taurates are capric ester taurate, cocoyl taurate, lauroyl taurate, myristoyl taurate, caproyl taurate, oleoyl taurate, capryloyl taurate, palmitoyl taurate, stearoyl taurate, linoleoyl taurate, salts of these and combinations thereof.

The N-alkyl acyl taurate surfactants herein are generally described by formula II illustrated below.

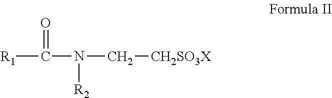

Where: R¹ is an alkyl group with 5 to 23 carbon atoms 7-21, 7-17, 7-15, 7-13, 11-17, 11-15, 11-13 or even 11 carbon atoms) and X is a suitable counterion (e.g., sodium, potassium, magnesium, ammonium or triethanolamine) and R₂ is an alkyl group with 1 to 4 carbon atoms. Some nonlimiting examples of N-alkyl acyl taurates that may be suitable for use herein include methyl capric ester taurate, methyl cocoyl taurate, methyl lauroyl taurate, methyl myristoyl taurate, methyl caproyl taurate, methyl oleoyl taurate, methyl capryloyl taurate, methyl palmitoyl taurate, methyl stearoyl taurate, methyl linoleoyl taurate, salts of these and combinations thereof.

It is to be appreciated that the taurate surfactants described herein are typically not single compounds, as suggested by their general formula (I) or (II), but rather a mixture of several homologs having varied chain lengths and molecular weights. Additionally, the taurate surfactants herein may be either saturated or unsaturated.

### Co-Surfactants

The personal care composition herein may include a co-surfactant selected from anionic surfactants, amphoteric surfactants, non-ionic surfactants and combinations of these. Some non-limiting examples of anionic co-surfactants include non-taurate, anionic surfactants such as isethionates, carboxylates, sulfonates (e.g., alpha olefin sulfonates, linear alkylbenzene sulfonates, alkyl glyceryl sulfonates, sodium laurylglucosides hydroxypropylsulfonate), branched alkyl sulfates, sulfosuccinates, sulfoacetates, sulfolaurates, amino acid-based surfactants (e.g., glycinates, sarcosinates, alaninates, glutamates), lactate- and lactylate-based surfactants (e.g., sodium lauroyl lactate and sodium lauroyl lactalyte), phosphate ester surfactants and combinations thereof.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. Some non-limiting examples of zwitterionic surfactants include amidosulfobetaines, hydroxysultaines, amidopropyl hydroxysultaines, and combinations thereof. Some non-limiting examples of amphoteric surfactants include amphoacetates, amphodiacetates, betaines, amidobetaines (e.g., cocamidopropyl betaine and lauramidopropyl betaine), amidosulfobetaines, propionates, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereof. Some particularly suitable examples of non-ionic surfactants include cocamide, cocamide MEA, PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US 2019/0105246, US 2018/0098923, U.S. Pat. No. 9,271,908, WO 2020/016097, and McCutcheon's Emulsifiers and Detergents, 2019, MC Publishing Co.

The cosurfactant may be present in the personal care compositions at 1% to 15% (e.g., 2-10%, 3-9%, 4-8%, or even 5-7%). The amount of co-surfactant in the composition can be important and should be tailored to balance viscosity building with cleaning and/or conditioning benefit. For example, too much amphoteric co-surfactant can make the surfactant system less salt tolerant and may impede the ability of the surfactant system to form a suitable coacervate upon dilution with water. This can be especially problematic when the composition contains a cationic polymer because the lowered salt tolerance of the surfactant system may cause the cationic polymer to precipitate out. In some embodiments, the composition may include a weight ratio of total taurate surfactant to amphoteric co-surfactant of 12:1 to 1:1 (6:1 to 3:10, 4:1 to 1:3, or even 2:1). It may be particularly desirable to provide a ratio of detersive surfactant to co-surfactant of 0.5:1 to 2:1 (e.g., 1:1 to 1.5:1).

### Cationic Polymer

The personal care compositions herein may include 0.05-3% of a cationic polymer (e.g., 0.1-2%, or even 0.2-0.8%) to provide improved appearance, feel or deposition benefits to hair or skin. The cationic polymer can have a weight average molecular weight of 50 kDa to about 5 MDa (e.g., 500 kDa-4 MDa, 1-3 MDa, 1.2-2 MDa, or even 1.4-1.8 MDa) and a charge density of 0.2 meq/g to 12 meq/g (e.g., 0.4-10 meq/g, 0.4-5 meq/g, 0.4-4 meq/g, 0.4-3 meq/g, or even 0.4-2 meq/g). The charge densities can be measured at the pH of intended use of the personal care composition, which can be pH 3 to pH 9 (e.g., pH 4-8 or pH 4.5-6.5).

The cationic polymers may include cationic, nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers, as long as the polymers remain soluble. Examples of suitable counterions include halide counterions (e.g., chloride, fluoride, bromide, iodide).

Some nonlimiting examples of cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some nonlimiting examples of cationic protonated amino and quaternary ammonium monomers include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (e.g., chloride salt) (referred to in the industry by the Personal Care Products Council (“PCPC”) as Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium-6 and Polyquaternium-7, respectively); amphoteric copolymers of acrylic acid including copolymers of acrylic acid and dimethyldiallylammonium chloride (Polyquaternium-22), terpolymers of acrylic acid with dimethyldiallylammonium chloride and acrylamide (Polyquaternium-39), and terpolymers of acrylic acid with methacrylamidopropyl trimethylammonium chloride and methylacrylate (Polyquaternium-47). In some aspects, suitable cationic substituted monomers include cationic substituted dialkylaminoalkyl acrylamides, dialkylaminoalkyl methacrylamides, and combinations thereof. The cationic polymer can be AM:TRIQUAT which is a copolymer of acrylamide and 1,3-Propanediaminium, N-[2-[[dimethyl[3-[(2-methyl-1-oxo-2-propenyl)amino]propyl]ammonio]acetyl]amino]ethyl]2-hydroxy-N,N,N′,N′,N′-pentamethyl-, trichloride (Polyquaternium-76). AM:TRIQUAT may have a charge density of 1.6 meq/g and a molecular weight of 1.1 MDa.

In some aspects, the cationic monomer can be polymethyacrylamidopropyl trimonium chloride, available under the trade name Polycare® 133, from Solvay (Brussels, Belgium). Copolymers of the cationic monomer may also suitable, and the charge density of the total copolymer can be 2.0 meq/g to 4.5 meq/g.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to in the industry (PCPC) as Polyquaternium-10 and available from Dow Chemical Company as UCARE™ JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as Polyquaternium-24.

Further examples of cationic polymers include cationic guar gum derivatives, such as guar hydroxypropyltrimonium chloride, such as the Jaguar® series available from Solvay and the N-Hance™ and AquaCat™ series from Ashland (Wilmington, Delaware). Additional disclosure of cationic guar gum derivatives can be found in U.S. Pat. No. 6,930,078.

In some instances, the cationic polymer may include a synthetic cationic polymer or derivative thereof present at 0.025% to about 5%. Preferred synthetic cationic polymers are generally water-soluble or dispersible and non-crosslinked. In some instances, the synthetic cationic polymer can be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in US 2003/0223951.

### Carrier

The composition may optionally include 20-95% of an aqueous carrier such as water and/or a water miscible solvent. The type and amount of aqueous carrier should be selected to provide the composition with the desired rheological properties. The liquid carrier can be water with, e.g., less than 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

### Optional Ingredients

The personal care compositions described herein may include a variety of optional ingredients to tailor the properties and characteristics of the composition, as desired. The optional ingredients may be materials that are commonly included in compositions of the type. The optional ingredients should be physically and chemically compatible with the essential components of the personal care composition and should not otherwise unduly impair the stability, aesthetics, or performance of the composition. Individual concentrations of optional components can generally range from 0.001% to 10%.

Some non-limiting examples of optional ingredients that can be included in the personal care compositions herein include deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide, 1,10-phenanthroline), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Method of Making a Personal Care Composition

The personal care composition described herein can be made using conventional methods for making compositions of the type desired (e.g., shampoo, conditioner or body wash). A particularly suitable method of making the compositions herein is described in Example 1 below. In some aspects, the composition may include a gel network to aid in the conditioning of hair or scalp. U.S. Publication No. 2006/269501 discloses methods of making gel networks that may be suitable for use herein.

### Method of Use

The personal care compositions described herein can be used in a conventional manner for cleansing and conditioning of hair or skin. Effective amounts of the composition for use generally range from 1 g to 50 g (e.g., 1 g to about 20 g). Generally, a method of treating hair or skin can include applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. The personal care composition can be used as a liquid, solid, semi-solid, flake, gel, foam, in a pressurized container with a propellant added, or used in a pump spray form. The viscosity of the product may be selected to accommodate the form desired.

In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### Methods

### Lather Robustness

Consumers commonly associate foaming and lathering with the quality of a personal cleansing composition such as a shampoo or body wash. This method provides a way to simulate the lather produced by surfactants under typical in-use conditions, across a broad range of water hardness conditions, and quantify certain lather properties. Oil (e.g., sebum) is one of the most common contaminates found on hair that can undesirably affect the lather properties of a shampoo. Thus, this method evaluates the effect of oil on lather properties.

100 mL of DI water, which has a water hardness of 0 gpg at 100° F., is added to a suitable blender (e.g., KitchenAid KSB560CU1 brand food mixer or equivalent), followed by 2 mL of the test composition and 1 mL of extra virgin olive oil. Blend the mixture on “stir” setting for 30 seconds and measure the height of the lather within the blender in centimeters and record as 0 gpg Lather Height.

100 mL of 25 gpg water, at 100° F., is added to a suitable blender (e.g., KitchenAid KSB560CU1 brand food mixer or equivalent), followed by 2 mL of the test composition and 1 ml of extra virgin olive oil. Blend the mixture on “stir” setting for 30 seconds and measure the height of the lather within the blender in centimeters and record as 25 gpg Lather Height. 25 gpg water can be made by first making a brine solution that contains 22.5 g magnesium chloride hexahydrate and 61 g calcium carbonate fully dissolved in 1 L DI water, then adding 235 mL of that brine solution to 50 L DI water and mixing thoroughly to ensure homogeneity.

The 25 gpg water can be prepared as follows. Dissolve 22.5 g of magnesium chloride hexahydrate and 61 g of calcium carbonate in 1 liter of DI water to form a brine solution. Add 235 mL of brine solution to 50 L of DI water and mix to provide a 25 gpg water solution.

The Lather Robustness of a composition, which is represented by the % drop in Lather Height between 0 gpg and 25 gpg water, can be calculated using the following equation:

$$\% drop = \frac{0 gpg Lather Height - 25 gpg Lather Height}{0 gpg Lather Height}$$

### Rheology

Personal care composition viscosities can be measured on a 2.5 mL sample using a cone and plate Brookfield® RS brand rheometer with cone C75-1 at 2 s⁻¹, 27° C. at 3 mins.

### Water Hardness Method

The water hardness of a sample of water can be determined by using a test strip from a water hardness test kit such as WaterWorks™ Total Hardness or equivalent according to the manufacturer's instructions for use.

### EXAMPLES

### Example 1: Example Formulations

Table 1 provides working examples of inventive personal care composition formulations. The compositions in Table 1 are made by adding DI water to a mixing vessel and then adding each subsequent ingredient while stirring. Surfactants with low water solubility such as cocamide MEA or sodium cocoyl isethionate, if present, require the composition to be heated to 50-75° C. and stirred until fully solubilized (i.e., no visible particles remain and batch is clear). The remaining ingredients, except for cationic polymer or volatile materials, are then added to the mixing vessel and mixed until fully dissolved or solubilized. If heated, the composition is then cooled to 35° C. or less before volatile ingredients such as perfume are added. If cationic polymer is present, in a separate container, the cationic polymer is mixed with water at a 1:20 ratio (polymer:water) to form a slurry or dilute solution, which is then added to the cooled composition in the mixing vessel and mixed for 10 minutes. The pH of the composition is adjusted with citric acid (typically, 0.2-0.5%). Viscosity is adjusted with sodium chloride. DI water is added to bring the final volume to 100%. The mixture is mixed until homogeneous (˜10 minutes).

The lather height of the example composition was determined according to the Lather Robustness method described above. A percent drop in lather height of more than 55% is considered unacceptable. A percent drop in lather height of 55% or less is considered acceptable, a percent drop of less than 30% is preferred, a percent drop of less than 20% is particularly preferred, and a percent drop of less than 10% is ideal.

TABLE 1

| | 1 | 2 | 3 | 4 | | |
|---|---|---|---|---|---|---|
| Ingredient | Active wt % | | | | 5 | 6 |
| SLT ¹ | 5.5 | 7.3 | 5 | 3.6 | 4.4 | 3.6 |
| SMLT ² | 0.5 | 0.7 | | 0.4 | 0.6 | 0.4 |
| Sodium C14-16 Olefin | 4 | 3 | 3 | 4 | | |
| Sulfonate ³ | | | | | | |
| Cocamidopropyl Betaine ⁴ | 5 | 4 | 5 | 2 | 2 | 4 |
| Lauramidopropyl Betaine ⁵ | | | | 2 | | |
| Ammonium lauryl sulfate ⁹ | | | | | 5 | 4 |
| Guar Hydroxypropyltrimonium | 0.4 | 0.4 | | 0.4 | | |
| Chloride ⁶ | | | | | | |
| Polyquaternium-10 ⁷ | | | 0.25 | | | |
| Piroctone Olamine ⁸ | | | | 0.5 | | |
| Sodium Benzoate | 0.5 | 0.5 | 0.75 | 0.15 | | |
| Sodium Salicylate | 0.25 | 0.25 | 0.45 | .015 | | |
| Tetrasodium EDTA | 0.13 | 0.13 | 0.16 | .013 | | |
| Perfume | 0.8 | 0.8 | 1.1 | 0.75 | | |
| Citric Acid | to pH | to pH | to pH | to pH | to pH | to pH |
| | 5.4 | 5.5 | 5.9 | 5.4 | 5.0-5.5 | 5.0-5.5 |
| Sodium Chloride (excludes | 0.2 | 0.6 | | 0.2 | 1.5 | 0.3 |
| carryover) | | | | | | |
| Water | QS | QS | QS | QS | QS | QS |
| Ratio of SLT to SMLT | 10.3:1 | 10.3:1 | N/A | 10.3:1 | 8:1 | 8:1 |
| total surfactant | 15 | 15 | 13 | 12 | 12 | 12 |
| Total taurate surfactant | 6 | 8 | 5 | 4 | 5 | 4 |
| Total amphoteric surfactant | 5 | 4 | 5 | 4 | 2 | 4 |
| Total Taurate:amphoteric ratio | 1.2:1 | 2:1 | 1:1 | 1:1 | 2.5:1 | 1:1 |
| lather height, 0 gpg (cm) | 8.5 | 8.5 | 8 | 8 | 9 | 7.9 |
| lather height, 25 gpg (cm) | 8 | 4 | 5.0 | 6.5 | 5.9 | 4.5 |
| % drop 0 to 25 gpg | 6% | 53% | 38% | 19% | 34% | 43% |

¹ Sodium Lauroyl Taurate from P&G Chemicals
² Sodium Methyl Lauroyl Taurate from P&G Chemicals or Geropon ® TL 32 L from Syensqo
³ Bio-terge ® AS-40 HP from Stepan
⁴ Tego ® Betain CK pH 12 from Evonik
⁵ Mirataine ® DAB ULS MB from Syensqo
⁶ Naternal ™ Excel from Syensqo
⁷ UCARE ™ JR30M from Dow
⁸ Octopirox ® from Clariant
⁹ Texapon ® ALS Benz from BASF

### Example 2: Formulation Examples

Table 2 shows prophetic examples of the inventive personal care composition. These examples can be made as described above using known techniques. Unless indicated otherwise, the materials used in these examples can be sourced from the same suppliers as described above in Example 1.

| | 7 | 8 | 9 | 10 | 11 | 12 |
|---|---|---|---|---|---|---|
| Ingredient | Active wt % | | | | | |
| SLT | 4.0 | 6.6 | 2.0 | 5.8 | | |
| SMLT | 4.0 | 0.4 | 0.5 | 0.2 | | |
| SCT ¹ | | | | | 2.3 | 9.3 |
| SMCT ² | | | | | 1.2 | 0.2 |
| Sodium C14-16 Olefin Sulfonate | | | | 4 | | |
| Sodium C12-14 Olefin Sulfonate | | | | | 4.5 | |
| Sodium Tridecyl Sulfate (80-99% | | 6 | 2.5 | | | |
| branched alkyl chain) | | | | | | |
| Lauryl Glucoside ³ | | | 1.2 | | | |
| Disodium laureth sulfosuccinate ⁴ | | | | | | 2 |
| Disodium lauryl sulfosuccinate ⁵ | | 2 | | | | |
| Cocamidopropyl betaine | 7 | | | | | |
| Lauramidopropyl betaine | 2.8 | | | 1 | | 2 |
| Coco-betaine ⁶ | | | | 3 | | |
| Lauryl betaine ⁷ | | | 1.75 | | 2 | 2 |
| Sodium lauroamphoacetate ⁸ | | | 1 | | | |
| Disodium cocoamphodiacetate ⁹ | | 4 | | | | 1 |
| Alpha-Glucan | | | 0.75 | | | 0.5 |
| hydroxypropyltrimonium chloride ¹⁰ | | | | | | |
| Guar Hydroxypropyltrimonium | | 0.6 | | | 0.2 | |
| Chloride | | | | | | |
| Guar Hydroxypropyltrimonium | | | | 0.5 | | 0.1 |
| Chloride ¹¹ | | | | | | |
| Polyquaternium-10 | 0.3 | | | | 0.2 | |
| Piroctone Olamine ¹² | 0.5 | | | | | |
| Zinc Pyrithione ¹³ | | | | 1 | | |
| Glycol Distearate ¹⁴ | | | | 2.5 | | |
| Sodium Benzoate | 0.15 | 0.18 | 0.1 | 0.25 | 0.5 | 0.25 |
| Sodium Salicylate | 0.15 | 0.2 | 0.1 | | | 0.2 |
| Tetrasodium EDTA | 0.13 | 0.13 | 0.13 | | | |
| Methylchloroisothiazolinone/ | | | | 0.0005 | 0.0005 | |
| Methylisothiazolinone ¹⁵ | | | | | | |
| Perfume | 1.1 | 0.5 | 0.8 | 1.2 | 1 | 1 |
| Citric Acid | to pH | to pH | to pH | to pH | to pH | to pH |
| | 6 | 5.5 | 4.7 | 5.2 | 6.5 | 5 |
| Sodium Chloride (excludes | 0.2 | 0.2 | 0.2 | 0.8 | 0.6 | 0.4 |
| carryover) | | | | | | |
| Water | QS | QS | QS | QS | QS | QS |

¹ Sodium cocoyl taurate from P&G Chemicals
² Sodium methyl cocoyl taurate from P&G Chemicals or Geropon ® TC 95 P or Geropon ® TC 30 from Solvay
³ Plantapon ® 1200 N UP from BASF
⁴ Chemccinate ™ DSLS-BA from Lubrizol
⁵ Cola ®Mate LA-40 from Colonial Chemical
⁶ Dehyton ® AB 30 from BASF
⁷ Empigen ® BB/HP from Innospec
⁸ Dehyton ® AB 30 from BASF (coco-betaine)
⁹ Amphosol ® 2C from Stepan
¹⁰ Alpha-Glucan Hydroxypropyltrimonium Chloride (MW = 185 kDA, DS = 0.03-0.15)
¹¹ N-Hance ™ BF-17 from Ashland
¹² Octopirox ® from Clariant.
¹³ U2 ZPT from Arxada or FPS ZPT from Kolon
¹⁴ EGDS Purified from Evonik
¹⁵ Kathon ™ CG broad-spectrum microbiocide

### Example 3: Lather Height Benefit

This Examples demonstrates the lather robustness benefit that can be provided by the inventive combination of acyl taurate and N-alkyl acyl taurate and amphoteric co-surfactant when the personal care composition is used in hard water. The lather heights of the test compositions were determined according to the Lather Robustness method. Inventive compositions are designated as “Inv” and comparative compositions (i.e., non-inventive) are designated as “Comp.”

TABLE 3

| | Comp 1 | Inv 1 | Comp 2 | Inv 2 | Comp 3 | Inv 3 | Comp 4 | |
|---|---|---|---|---|---|---|---|---|
| Ingredient | Active wt % | | | | | | | Inv 4 |
| SLT | 8.2 | 5.5 | 8.2 | 5.5 | 8.2 | 5.5 | 8.2 | 5.5 |
| SMLT | 0.8 | 0.5 | 0.8 | 0.5 | 0.8 | 0.5 | 0.8 | 0.5 |
| Cocamidopropyl Betaine | 3 | 6 | | | | | | |
| Lauramidopropyl Betaine | | | 3 | 6 | | | | |
| Coco-betaine | | | | | 3 | 6 | | |
| Sodium | | | | | | | 3 | 6 |
| Lauroamphoacetate | | | | | | | | |
| Citric Acid | to pH | to pH | to pH | to pH | to pH | to pH | to pH | to pH |
| | 5.0-5.5 | 5.0-5.5 | 5.0-5.5 | 5.0-5.5 | 5.0-5.5 | 5.0-5.5 | 5.0-5.5 | 5.0-5.5 |
| Sodium Chloride | | | | | | | 0.55 | |
| Water | QS | QS | QS | QS | QS | QS | QS | QS |
| Ratio of SLT to SMLT | 10.5:1 | 10.5:1 | 10.5:1 | 10.5:1 | 10.5:1 | 10.5:1 | 10.5:1 | 10.5:1 |
| total surfactant | 12 | 12 | 12 | 12 | 12 | 12 | 12 | 12 |
| Total taurate surfactant | 9 | 6 | 9 | 6 | 9 | 6 | 9 | 6 |
| Total amphoteric | 3 | 6 | 3 | 6 | 3 | 6 | 3 | 6 |
| surfactant | | | | | | | | |
| Total taurate:amphoteric | 3:1 | 1:1 | 3:1 | 1:1 | 3:1 | 1:1 | 3:1 | 1:1 |
| ratio | | | | | | | | |
| lather height, 0 gpg (cm) | 9 | 7.7 | 9 | 8.5 | 9 | 7.7 | 8 | 8 |
| lather height, 25 gpg (cm) | 0.2 | 6.8 | 0.5 | 7.0 | 0.1 | 6.8 | 2 | 5 |
| % drop 0 to 25 gpg | 98% | 12% | 94% | 18% | 99% | 12% | 75% | 38% |

As can be seen in Table 3, tailoring the ratio of the taurate to amphoteric is important for providing a hard water lather benefit. In particular, going from a 3:1 ratio, where there is essentially no lather and a 75-99% drop in lather height, to a 1:1 ratio provides a surprising improvement in hard water lather amount and substantially decreases the percent drop in lather.

### Example 4: Effect of Additional Co-Surfactants

This example demonstrates the criticality of the total taurate to amphoteric surfactant ratio even in the presence of additional co-surfactants on the hard water lather benefit of the personal care composition. Example Inv 3b was included to demonstrate that the ratio of acyl taurate to N-alkyl acyl taurate is not as critical for robust lather generation in hard water as it is for other composition properties such as acyl taurate solubility or viscosity building.

TABLE 4

| | Comp 1 | Inv 1 | Comp 2 | Inv 2 | Comp 3 | Inv 3a | Inv 3b |
|---|---|---|---|---|---|---|---|
| Ingredient | Active wt % | | | | | | |
| SLT | 4.4 | 3.6 | 4.4 | 3.6 | 4.4 | 3.6 | 3 |
| SMLT | 0.6 | 0.4 | 0.6 | 0.4 | 0.6 | 0.4 | 1 |
| Decyl glucoside | 5 | 4 | 5 | 4 | | | |
| Sodium C14-16 Olefin | | | | | 5 | 4 | 4 |
| Sulfonate | | | | | | | |
| Cocamidopropyl Betaine | 2 | 4 | | | 2 | 4 | 4 |
| Lauramidopropyl Betaine | | | 2 | 4 | | | |
| Citric Acid | to pH | to pH | to pH | to pH | to pH | to pH | to pH |
| | 5.0-5.5 | 5.0-5.5 | 5.0-5.5 | 5.0-5.5 | 5.0-5.5 | 5.0-5.5 | 5.0-5.5 |
| Sodium Chloride | 3.8 | 1.7 | 4.2 | 3.4 | 3.6 | 2.1 | 0.8 |
| Water | QS | QS | QS | QS | QS | QS | QS |
| Ratio of SLT to SMLT | 8:1 | 8:1 | 8:1 | 8:1 | 8:1 | 8:1 | 3:1 |
| Total surfactant | 12 | 12 | 12 | 12 | 12 | 12 | 12 |
| Total taurate surfactant | 5 | 4 | 5 | 4 | 5 | 4 | 4 |
| Total amphoteric | 2 | 4 | 2 | 4 | 2 | 4 | 4 |
| surfactant | | | | | | | |
| Total Taurate:amphoteric | 2.5:1 | 1:1 | 2.5:1 | 1:1 | 2.5:1 | 1:1 | 1:1 |
| ratio | | | | | | | |
| lather height, 0 gpg (cm) | 7.9 | 9 | 7.9 | 8.1 | 8.1 | 9 | 7.5 |
| lather height, 25 gpg (cm) | 0.9 | 6.8 | 0.5 | 7.2 | 1.1 | 6.8 | 5.5 |
| % drop 0 to 25 gpg | 89% | 25% | 94% | 11% | 86% | 25% | 27% |

| | Comp 4 | Inv 4 | Comp 5 | Inv 5 | Comp 6 | Inv 6 | Comp 7 | |
|---|---|---|---|---|---|---|---|---|
| Ingredient | Active wt % | | | | | | | Inv 7 |
| SLT | 4.4 | 3.6 | 4.4 | 3.6 | 4.4 | 3.6 | 4.4 | 3.6 |
| SMLT | 0.6 | 0.4 | 0.6 | 0.4 | 0.6 | 0.4 | 0.6 | 0.4 |
| Decyl glucoside | | | | | 5 | 4 | | |
| Sodium C14-16 Olefin | 5 | 4 | | | | | 5 | 4 |
| Sulfonate | | | | | | | | |
| Sodium C12-13 alkyl | | | 5 | 4 | | | | |
| sulfate ¹ | | | | | | | | |
| Cocamidopropyl Betaine | | | 2 | 4 | 2 | 4 | | |
| Lauramidopropyl Betaine | 2 | 4 | | | | | 2 | 4 |
| Piroctone olamine | | | | | 0.5 | 0.5 | 0.5 | 0.5 |
| Perfume | | | | | 0.75 | 0.75 | 0.75 | 0.75 |
| Citric Acid | to pH | to pH | to pH | to pH | to pH | to pH | to pH | to pH |
| | 5.0-5.5 | 5.0-5.5 | 5.0-5.5 | 5.0-5.5 | 5.0-5.5 | 5.0-5.5 | 5.0-5.5 | 5.0-5.5 |
| Sodium Chloride | 4.1 | 3.1 | 1.3 | 0.4 | 0.9 | 0.1 | 1.9 | 1.3 |
| Water | QS | QS | QS | QS | QS | QS | QS | QS |
| Ratio of SLT to SMLT | 8:1 | 8:1 | 8:1 | 8:1 | 8:1 | 8:1 | 8:1 | 8:1 |
| Total surfactant | 12 | 12 | 12 | 12 | 12 | 12 | 12 | 12 |
| Total taurate surfactant | 5 | 4 | 5 | 4 | 5 | 4 | 5 | 4 |
| Total amphoteric | 2 | 4 | 2 | 4 | 2 | 4 | 2 | 4 |
| surfactant | | | | | | | | |
| Total Taurate:amphoteric | 2.5:1 | 1:1 | 2.5:1 | 1:1 | 2.5:1 | 1:1 | 2.5:1 | 1:1 |
| ratio | | | | | | | | |
| lather height, 0 gpg (cm) | 8.1 | 9 | 9 | 9 | 9.0 | 8.8 | 9.0 | 9.0 |
| lather height, 25 gpg (cm) | 1.1 | 6.8 | 2.3 | 4.5 | 1.4 | 6.8 | 1.8 | 7.7 |
| % drop 0 to 25 gpg | 86% | 25% | 75% | 50% | 84% | 23% | 80% | 14% |

| | Inv 8 | Inv 9 | Inv 10 |
|---|---|---|---|
| Ingredient | Active wt % | | |
| SLT | 2.7 | 2.7 | 4.6 |
| SMLT | 0.3 | 0.3 | 0.4 |
| Sodium C14-16 Olefin Sulfonate | 7 | 5 | 3 |
| Cocamidopropyl Betaine | 5 | 5 | 5 |
| Guar Hydroxypropyltrimonium Chloride | 0.4 | 0.4 | 0.4 |
| Sodium Benzoate | 0.5 | 0.5 | 0.5 |
| Sodium Salicylate | 0.25 | 0.25 | 0.25 |
| Tetrasodium EDTA | 0.13 | 0.13 | 0.13 |
| Perfume | 0.8 | 0.8 | 0.8 |
| Citric Acid | to pH 5.4 | to pH 5.6 | to pH 5.6 |
| Sodium Chloride (excludes carryover) | 0.2 | 0.2 | 0.2 |
| Water | QS | QS | QS |
| Ratio of SLT to SMLT | 10.3:1 | 10.3:1 | 10.3:1 |
| total surfactant | 15 | 13 | 13 |
| Total Taurate surfactant | 3 | 3 | 5 |
| Total amphoteric surfactant | 5 | 5 | 5 |
| Total Taurate:amphoteric ratio | 0.6:1 | 0.6:1 | 1:1 |
| lather height, 0 gpg (cm) | 8 | 8.5 | 8.5 |
| lather height, 25 gpg (cm) | 7 | 5.5 | 7.5 |
| % drop: 0 to 25 gpg | 13% | 35% | 12% |

¹ Dacpon 27-23 AL from Sasol (54% branched alkyl chain)

As can be seen in Table 4, the comparative examples demonstrate that adding a third surfactant, (e.g., non-ionic or anionic co-surfactant), did not improve hard water lather when the ratio of total taurate to amphoteric surfactant was at 2.5:1, despite the taurate to non-ionic or anionic surfactant ratio being at 1:1. However, decreasing the ratio of taurate surfactant to amphoteric surfactant to 1:1 once again provided a surprising improvement in hard water lather and decreased % drop in lather height in the respective inventive compositions.

Perhaps even more surprising is that tailoring the ratio of taurate to amphoteric surfactant was found to improve hard water lather as much as increasing total surfactant level, a known compensation for the negative impact of hard water on lather amounts. For example, Composition Inv 9 contains 13% total surfactant and exhibited a percent drop in lather height of 35%, which is acceptable. Comparing Composition Inv 9 to Inv 8 shows that adding 2% sodium C14-16 olefin sulfonate to Composition Inv 9 and thus increasing the total surfactant level to 15% (Inv 8) resulted in a percent drop improvement to 13%. Increasing the total taurate to amphoteric surfactant ratio from 0.6:1 (Inv 9) to 1:1 (Inv 10) while maintaining a low 13% total surfactant level provided an unexpected percent drop improvement to 12%, which was equivalent to the improvement observed from increasing total surfactant levels. This further highlights the importance of the ratio of taurate to amphoteric surfactant to provide hard water lather benefits even at low total surfactant levels.

### Example 5: Effect of Cationic Polymer

This example demonstrates the effect of a cationic polymer such as guar hydroxypropyltrimonium chloride or polyquaternium-10 on the hard water lather benefit of the personal care composition. Inventive examples of personal care compositions are provided in Table 5 along with a summary of the lather test results.

TABLE 5

| | 5-1 | 5-2 | 5-3 | 5-4 |
|---|---|---|---|---|
| Ingredient | Active wt % | | | |
| SLT | 9.1 | | 4.1 | 6 |
| SMLT | 0.9 | | 0.4 | |
| SCT | | 8.2 | | |
| SMCT | | 0.8 | | |
| Coco-betaine | 6 | | | |
| Lauramidopropyl Betaine | | 7 | 5.4 | 5.4 |
| Cocamidopropyl Betaine | | | 4.4 | 4.4 |
| Guar | 0.4 | | | |
| Hydroxypropyltrimonium | | | | |
| Chloride | | | | |
| Polyquaternium-10 | | | 0.4 | 0.4 |
| Sodium Benzoate | | 0.25 | 0.75 | 0.75 |
| Sodium Salicylate | | 0.25 | 0.45 | 0.45 |
| Tetrasodium EDTA | 0.13 | 0.13 | 0.13 | 0.13 |
| Methylchloroisothiazolinone/ | 5 ppm | | | |
| Methylisothiazolinone | | | | |
| Perfume | 1.1 | 1 | 1.1 | 1.1 |
| Citric Acid | to | to | to | to |
| | pH 5.5 | pH 5.6 | pH 6.0 | pH 6.0 |
| Sodium Chloride | 0.4 | 1 | | |
| Sodium Xylene Sulfonate | | | | 0.5 |
| Water | QS | QS | QS | QS |
| Ratio of acyl taurate to | 10.3:1 | 10.3:1 | 10.3:1 | N/A |
| N-alkyl acyl taurate | | | | |
| Total surfactant | 16.0 | 16 | 14.3 | 15.8 |
| Total taurate surfactant | 10 | 9 | 4.5 | 6 |
| Total amphoteric surfactant | 6 | 7 | 9.8 | 9.8 |
| Total taurate:amphoteric ratio | 1.7 | 1.3 | 0.5 | 0.6 |
| lather height, 0 gpg (cm) | 9 | 8.5 | 7 | 8.5 |
| lather height, 25 gpg (cm) | 8.3 | 8 | 5.5 | 6.0 |
| % drop 0 to 25 gpg | 8% | 6% | 21% | 29% |

As can be seen in Table 5, the personal care composition still exhibited an acceptable amount of lather generation even when a cationic polymer was added.

### Example 6: Comparative Examples

This example provides Comparative personal care compositions that do not provide the hard water lather benefit of the compositions described herein.

TABLE 6

| | Comp | Comp | Comp | Comp | Comp | Comp |
|---|---|---|---|---|---|---|
| Ingredient | 6-1 | 6-2 | 6-3 | 6-4 | 6-5 | 6-6 |
| SLT | 7.695 | 0.855 | 7.695 | 0.855 | 8.2 | 5.5 |
| SMLT | 0.855 | 7.695 | 0.855 | 7.695 | 0.8 | 0.5 |
| Cocamidopropyl | 6.6 | 6.6 | 6.6 | 6.6 | | |
| hydroxysultaine (CAPHS) ¹ | | | | | | |
| Lauryl hydroxysultaine ² | | | | | 3 | 6 |
| Cocamidopropyl betaine | | | | | | |
| Polyquaternium-7 ³ | 0.03 | 0.03 | 0.03 | 0.03 | | |
| PEG-150 distearate ⁴ | 0.2 | 0.2 | | | | |
| PEG-100 stearate ⁵ | | | 0.2 | 0.2 | | |
| Citric Acid | to pH | to pH | to pH | to pH | to pH | to pH |
| | 5.0-5.5 | 5.0-5.5 | 5.0-5.5 | 5.0-5.5 | 5.0-5.5 | 5.0-5.5 |
| Sodium chloride | | | | | | |
| Water | QS | QS | QS | QS | QS | QS |
| Ratio of SLT to SMLT | 9:1 | 1:9 | 9:1 | 1:9 | 10.5:1 | 10.5:1 |
| Total surfactant | 15.15 | 15.15 | 15.15 | 15.15 | 12 | 12 |
| Total taurate surfactant | 8.55 | 8.55 | 8.55 | 8.55 | 9 | 6 |
| Total zwitterionic surfactant | 6.60 | 6.6 | 6.6 | 6.6 | 3 | 6 |
| Total amphoteric surfactant | 0 | 0 | 0 | 0 | 0 | 0 |
| Total taurate:total | 1.3:1 | 1.3:1 | 1.3:1 | 1.3:1 | 3:1 | 1:1 |
| amphoteric/zwitterionic ratio | | | | | | |
| lather height, 0 gpg (cm) | 7.8 | 8.0 | 8.0 | 8.0 | 8.6 | 8.6 |
| lather height, 25 gpg (cm) | 2 | 3.5 | 2 | 3 | 0.1 | 0.1 |
| % drop 0 to 25 gpg | 74% | 56% | 75% | 63% | 99% | 99% |

¹ StarSurfTM CAPHS from StarChem.
² Mirataine ® LHS from Syensqo
³ Merquat ™ 550 PR Polymer from Lubrizol
⁴ Hallstar ® PEG 6000 DS from Hallstar
⁵ Hallstar ® PEG 4400 MS MB from Hallstar

As can be seen in Table 6, the comparative personal care compositions do not provide an acceptable amount of lather robustness.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous personal care composition, comprising:
a) a detersive surfactant comprising an acyl taurate surfactant or a combination of an acyl taurate and an N-alkyl acyl taurate surfactant;
b) an amphoteric co-surfactant, wherein a ratio of detersive surfactant to co-surfactant is about 0.5:1 to about 2:1; and
c) water.

2. The personal care composition of claim 1, wherein the composition exhibits less than a 55% decrease in lather according to the Lather Robustness Test.

3. The personal care composition of claim 1, wherein the amphoteric surfactant is selected from alkylamidopropyl betaines, alkyl betaines, amphoacetates and combinations thereof.

4. The personal care composition of claim 3, wherein the amphoteric surfactant is selected from cocamidopropyl betaine, lauramidopropyl betaine, coco-betaine, lauryl betaine, cetyl betaine, sodium lauroamphoacetate, sodium cocoamphoacetate, disodium lauroamphodiacetate, disodium cocoamphodiacetate and combinations thereof.

5. The personal care composition of claim 1, further comprising a non-ionic surfactant.

6. The personal care composition of claim 1, further comprising an anionic co-surfactant.

7. The personal care composition of claim 1, wherein the composition comprises a combination of acyl taurate and N-acyl alkyl taurate at a weight ratio of acyl taurate to N-alkyl acyl taurate of about 1:1 to about 50:1.

8. The personal care composition of claim 7, wherein the weight ratio of acyl taurate to N-alkyl acyl taurate is about 2:1 to about 25:1

9. The personal care composition of claim 1, wherein the acyl taurate surfactant is a C8-C24 acyl taurate surfactant.

10. The personal care composition of claim 9, wherein the C8-C24 acyl taurate surfactant is selected from the group consisting of capric ester taurate surfactants, cocoyl taurate surfactants, lauroyl taurate surfactants, myristoyl taurate surfactants, caproyl taurate surfactants, oleoyl taurate surfactants, capryloyl taurate surfactants, palmitoyl taurate surfactants, stearoyl taurate surfactants, linoleoyl taurate surfactants, salts of these and combinations thereof.

11. The personal care composition of claim 1, wherein the N-alkyl acyl taurate surfactant is selected from methyl substituted acyl taurates, ethyl substituted acyl taurates, propyl substituted acyl taurates, butyl substituted acyl taurates, salts of these and combinations thereof.

12. The personal care composition of claim 11, wherein the N-alkyl acyl taurate is selected from the group consisting of methyl capric ester taurate, methyl cocoyl taurate, methyl lauroyl taurate, methyl myristoyl taurate, methyl caproyl taurate, methyl oleoyl taurate, methyl capryloyl taurate, methyl palmitoyl taurate, methyl stearoyl taurate, methyl linoleoyl taurate, salts of these and combinations thereof.

13. The composition of claim 1, further comprising an additional ingredient selected from deposition aids, conditioning agents, anti-dandruff agents, anti-microbial agents, suspending agents, viscosity modifiers, dyes, pigments, nonvolatile solvents, diluents, pearlescent aids, foam boosters, pH adjusting agents, perfumes, preservatives, chelants, proteins, vitamins, amino acids, skin active agents, sunscreens, UV absorbers, stabilizers, and combinations of these.

14. The composition of claim 13, wherein the composition comprises an anti-dandruff agent selected from the group consisting of azoles, hydroxyl pyridones, zinc pyrithone, zinc carbonate, piroctone olamine, piroctone, sulfur, ciclopirox, rilopirox, MEA-hydroxyoctyloxypyridinone, salicylic acid, azoxystrobin, 1,10-phenanthroline and combinations thereof.

15. The composition of claim 1, wherein the composition is substantially free of sulfated surfactants.

16. The composition of claim 1, further comprising about 0.05% to about 3% of a cationic polymer that has a weight average molecular weight of about 50 kDa to about 5 MDa and a charge density of about 0.2 meq/g to about 12 meq/g.

17. A method of cleaning a bodily surface, comprising:
a) applying water to a target bodily surface where cleaning is desired, wherein the water has a hardness value of more than 3 grains per gram of water according to the Water Hardness method;
b) applying the composition of claim 1 to the target bodily surface; and
c) rinsing the personal cleansing composition off the bodily surface with the water.

18. The method of claim 17, wherein the target bodily surface is hair or skin.

19. The method of claim 17, wherein the personal care composition is a shampoo, conditioner or body wash.
